(19) United States
(12) Patent Application Publication
(10) Pub. No.: US 2012/0109028 A1
(43) Pub. Date: May 3, 2012

(54) Obstetric Birthing Assistant

(76) Inventor: Cynthia Sheffield, Mt. Gilead, NC (US)

(21) Appl. No.: 13/153,518

(22) Filed: Jun. 6, 2011

Related U.S. Application Data
(60) Provisional application No. 61/409,777, filed on Nov. 3, 2010.

(51) Int. Cl. A61B 17/42

(52) U.S. Cl. 601/45

(57) ABSTRACT

An obstetrical assistive device is provided for facilitating “pushing” during childbirth. The device comprises a pair of handlebar assemblies conjoined in the center by a ring. The handlebars may be constructed of durable material such as plastic or rubber and may have a soft, high-friction material covering the gripping area. The center ring may be constructed of any high tension bearing material like rope, rubber or metal. A patient may use the device by herself or in conjunction with another person. To use the device with another person, the patient and other user grasp opposing ends of the device and exert retractive force. Alternatively, the device may be secured to a support bar by its center ring so that a patient may pull on a handlebar without having another user grasping the opposing handlebar.

### CROSS REFERENCE TO RELATED APPLICATION

This application claims the benefit of U.S. Provisional Application No. 61/409,777 filed on Nov. 3, 2010, entitled “Sheffield Delivery Assistant.”

### BACKGROUND OF THE INVENTION

1. Field of the Invention

The present invention relates to an obstetrical tool and more specifically to a tension providing device for assisting with the birthing process.

During the process of childbirth it is necessary for a woman to exert pushing motions on her abdomen by flexing her muscles. This flexing motion pushes the baby down from the uterus into the birthing canal where it can then be born. Some women lack the abdominal strength required to push a baby downward. Other women are given an epidural shot to numb the pain and are therefore not able to push hard enough because they are unable to feel their abdominal muscles flex. To assist with the pushing motions, a woman giving birth may pull rigorously on an object. The act of pulling an object towards herself forces a woman's abdominal muscles to flex, facilitating stronger “pushes”.

Hospital nurses and midwives often utilize a towel by having the patient grasp one end while the nurse grabs the other; they then pull in opposite directions. Other methods of pulling include the use of rope tugs, some of which use wrist straps. These methods are potentially hazardous since rope is not sanitizable or waterproof and can allow harmful bacteria to grow on its surface. Additionally, the use of wrist straps is problematic because of the strain it can place on a woman's wrist joint. Such strain can lead to serious wrist injuries like torn ligaments, bone breaks, and potential dislocation of joints.

Towels and rope tugs are also problematic because they require both users to be engaged in pulling at all times. This prevents a nurse from completing tasks in preparation for the birth or from checking on a woman's medical statistics to ensure her safety. A pulling device is needed that may be used by a patient by herself or in conjunction with a nurse, so that a patient may continue to use the device while a nurse tends to other duties.

2. Description of the Prior Art

The prior art contains a variety of handled pulling devices for providing tension between a user and a person or object. These devices have familiar design and structural elements for the purposes of promoting abdominal flexing by providing resistive force; however they are not adapted for providing a user with the ability to use the device individually or with another person.

Freymann, U.S. Pat. No. 2,009,655 discloses an obstetrical device. It is held in the hands of a patient who is in the process of giving birth to a child. An attached pair of straps adapted to be secured to the foot of the table or bed on which the patient reclines. Said straps stabilize the physical force exerted by the arms while pulling rearwardly upon the straps. Freymann does not disclose a pair of handlebars, or a tension bearing ring providing for single or multi-person use.

Balne, U.S. Pat. No. 3,068,002 discloses a push and pull type of exercise apparatus. The apparatus having at least one handle at one end of a tow rope, and at least one handle attached to the opposite end of said rope. In an alternate embodiment the device may have at least one handle secured to a first end of the tow rope and a foot engaging assembly secured to the opposing end of said tow rope. The separate embodiments provide for single or multi-person. Balne does disclose an apposed pair of handlebars, or a tension bearing ring providing for single or multi-person use within the same embodiment of the device.

Holland, U.S. Pat. No. 4,043,290 discloses a tow rope handle for water skiing comprising an elongated hand grip with a pair of spaced holes extending transversely therethrough, a tow line threaded through each spaced hole and formed in a loop to firmly encircle the hand grip and a protective sleeve encompassing each tow line directly adjacent to the hand grip. Holland does not disclose an apposed pair of handlebars, or a tension bearing ring providing for single or multi-person use.

Hayden, U.S. Pat. No. 4,392,833 discloses a water ski tow bar device. The device may be arranged to form a single tow bar and is separable into two tow bars or handles which may be used by a skier being pulled by a boat. Hayden does not disclose an apposed pair of handlebars, or a tension bearing ring providing for single or multi-person use.

Taylor, U.S. Pat. No. 4,540,371 discloses a ski rope handle assembly. The device includes a double entry figure eight loop connection formed about each end of a ski rope handle. Alternatively the device may include an external rope loop connection that is continuously wound in an arcuate fashion about each end of the ski rope handle after which the open, wound loop is directed such that a forward extending portion of the rope adjacent the loop is threaded through an opening formed within an adjacent finger protector sleeve. This handle and tow rope combination is used for the purposes of pulling a water skier behind a boat or the like. Taylor does not disclose an apposed pair of handlebars, or a tension bearing ring providing for single or multi-person use.

Benson, U.S. Pat. No. 7,318,810 discloses a device for assisting in the active and pushing stages of childbirth. The child birthing device consists of a first loop that can be used to provide resistance for pulling. The first loop is connected by means of a length of rope to a braided handhold two wrist loops attached thereto. A pregnant person can then insert her hands through the wrist loops and grasp the braided handhold. She can then pull against tension that is applied to the other end of the rope. The pulling action while pushing redirects where the pushing takes place in the pregnant persons body and increases the effectiveness of her “pushing”. Benson does not disclose an apposed pair of handlebars, or a tension bearing ring providing for single or multi-person use.

The devices disclosed by the prior art do not address the need for single or multi-person use. The current invention relates to a device for assisting a woman during childbirth by providing resistance to her while she pulls on a handle. It substantially diverges in structural elements from the prior art, consequently it is clear that there is a need in the art for an improvement to the existing obstetrical pulling devices. In this regard the instant invention substantially fulfills these needs.

### SUMMARY OF THE INVENTION

In view of the foregoing disadvantages inherent in the known types of obstetrical pulling devices now present in the prior art, the present invention provides a new multi-user or single user capability wherein the same can be utilized for providing convenience for a user giving childbirth who may or may not have a nurse available to exert tension on the opposing side of the user's device. The obstetrical pulling device comprises two handles joined together by a ring several inches in diameter. Said handles are similar to those used by water skiers and are adapted to allow a user to grip the handle with both hands. To prevent injuries cause by abrasive materials such as rope, the handles should be constructed of a durable material such as plastic or rubber, and covered in a soft, high-friction material. The selected material must be suitable for sterilization to avoid exposing patients to bacteria buildup.

A ring conjoining the two handles bears the tension between two users pulling on the device's handles, or a single user pulling on a handle while the device is connected to a support bar. Said ring may be constructed of any strong, durable material that is capable of handling tension, such as rope, flexible plastic, or rubber. The device may be used by a patient who grips one handle, and a nurse who grips the opposing handle. Both users exert backward force on the handles with the intended result of forcing the patient to exert abdominal pressure on a baby during birth. If the nurse is unavailable to assist in operating the device, it may be secured to a support bar by the middle ring. A patient then grips either handle of the device and pulls on it by herself to create abdominal pressure.

An object of the present invention to provide a new and improved obstetrical pulling device having all of the advantages of the prior art and none of the disadvantages.

Another object of the present invention is to provide a new and improved obstetrical pulling device that may be operated by a nurse and a patient pulling on opposing ends of the device, or alternatively may be operated by a patient pulling on the device which is secured by a center ring to a birthing bar.

Yet another object of the present invention is to provide a new and improved obstetrical pulling device having two handles made of a sanitizable material to reduce a patient's exposure to bacteria.

Still another object of the present invention is to provide a new and improved obstetrical pulling device having handles covered in a material providing a soft, but high-friction gripping surface to prevent abrasion to the hands of a user.

A further object of the present invention is to provide a new and improved obstetrical pulling device having resilient and durable construction.

Other objects, features and advantages of the present invention will become apparent from the following detailed description taken in conjunction with the accompanying drawings.

### BRIEF DESCRIPTIONS OF THE DRAWINGS

The above invention will be better understood and the objects set forth above as well as other objects not stated above will become more apparent after a study of the following detailed description thereof. Such description makes use of the annexed drawings wherein:

FIG. 1 shows a perspective view of an obstetrical pulling device according to the present invention.

FIG. 2 shows a perspective view of an obstetrical pulling device according to the present invention while in use. Two users are shown exerting retractive force on the device's handles.

FIG. 3 shows a perspective view of an obstetrical pulling device according to the present invention, while in use. A patient in the process of giving birth grips the device's handle and exerts retractive force on the device while it is removably affixed to a support bar.

### DETAILED DESCRIPTION OF THE INVENTION

Reference is made herein to the attached drawings. Like reference numerals are used throughout the drawings to depict like or similar elements of the obstetrical pulling device. For the purposes of presenting a brief and clear description of the present invention, the preferred embodiment will be discussed as used for providing assistance to patients giving birth. This is for representative purposes only and should not be considered to be limiting in any respect.

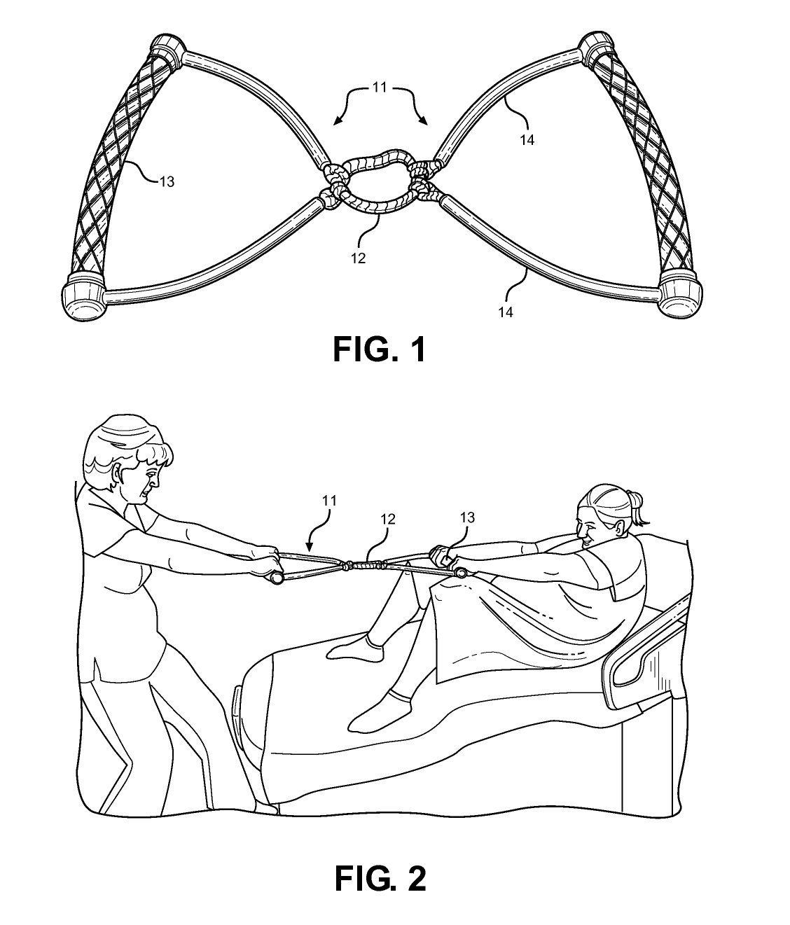

Referring now to FIG. 1, there is shown an obstetrical pulling device according to the present invention. The device comprises a pair of handle assemblies 11 conjoined at their middle by a tension bearing ring 12. Said handlebar assembly 11 includes a handle grip portion 13 fixedly secured on either end to a pair of handle supports 14. The handle grip 13 has a length adapted to allow two adult female hands to hold it. To reduce slipping of the hands and potential injuries due to abrasion the handle grip 13 may be covered in a soft, high-friction material such as rubber. The handle supports 14 have end caps disposed at their outer ends that include a recess adapted to snuggly house ends of the handle grip 13. The sides of said handle supports 14 extend arcuately inward towards said tension bearing ring 12. Any durable, waterproof, sterilizable material may be used in the construction of the handle grip 13 and handle supports 12, such as rubber or plastic.

In the center of the device, disposed between the pair of handle assemblies 11 is a tension bearing ring 12. Said tension bearing ring 12 is four to five inches in diameter and may be constructed of any material capable of bearing high levels of tension, such as rope, rubber, or metal. The inner ends of the handle supports are connected to the ring by small loops of rope secured to the former. Alternatively there may be cut-outs at the end of the handle supports adapted to allow the tension bearing ring 12 to pass through them, thus securing said ring to the handle supports 14.

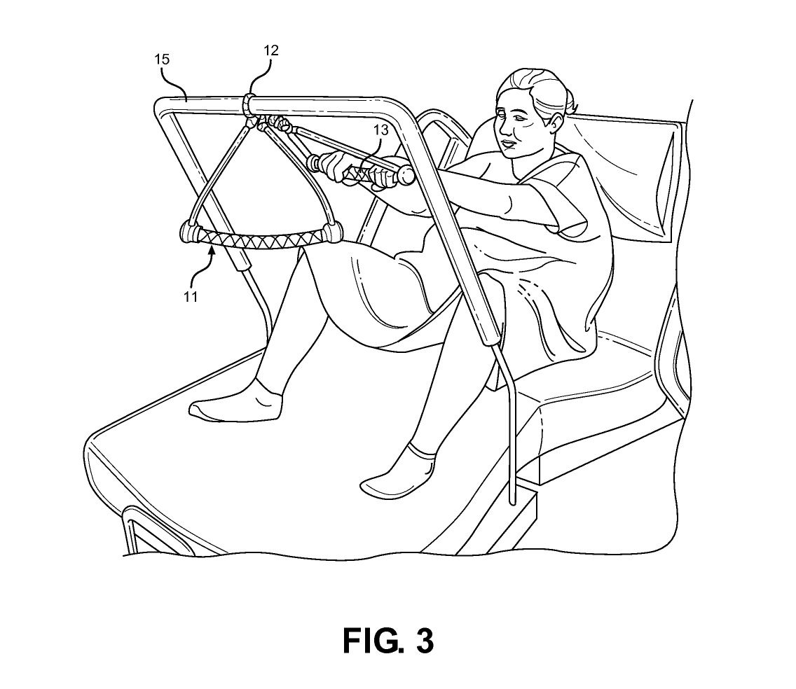

Referring now to FIGS. 2 & 3, there are shown two views of the present obstetrical pulling device in use. In FIG. 2, a nurse and patient lying on a hospital bed grip opposing ends of the device by its handle grips 13. The nurse and patient then exert retractive force against the device. Tension is distributed across the center ring 12 as the users pull on each handlebar assembly 11. In FIG. 3 a patient is shown lying on a hospital bed while firmly gripping the device by a handle grip 13. The center tension bearing ring 12 of the device is removably secured to a support bar 15. To use the device, the patient exerts retractive force against the support bar 15 by pulling on the handlebar assembly 11. In both forms of use, the force exerted on device by a patient results in abdominal pressure, which pushes an unborn baby downward towards the birthing canal. In this manner the device assists patients in the birthing process with regard to pushing the child into position to be delivered.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim the following:

1) An obstetrical assistive device for inducing abdominal flexing, comprising:
a pair of handlebar assemblies having handle grips and a pair of handle supports, said handle supports having end caps disposed at their outer ends, adapted to receive and permanently retain end portions of said handle grip,
a ring conjoining said handlebar assemblies at inner ends of their handle supports.

2) The device of claim 1, wherein said handle grip is covered in a soft, high-friction material.

3) The device of claim 1, wherein said ring is constructed of rope.

4) The device of claim 1, wherein said handlebar assemblies are constructed of a waterproof, sanitizable material.
